(19) United States
(12) Patent Application Publication
(10) Pub. No.: US 2019/0365939 A1
(43) Pub. Date: Dec. 5, 2019

(54) MICROBIAL CONTROL SYSTEM

(71) Applicant: SMARTWASH SOLUTIONS, LLC, Salinas, CA (US)

(72) Inventors: Christopher Michael MCGINNIS, Salinas, CA (US); James M. BRENNAN, Pleasanton, CA (US); Eric Child WILHELMSEN, Milpitas, CA (US); Craig Seward Tedmon, III, Marina, CA (US)

(21) Appl. No.: 16/418,737

(22) Filed: May 21, 2019

Related U.S. Application Data
(60) Provisional application No. 62/678,645, filed on May 31, 2018.

(51) Int. Cl. A61L 2/20; A61L 2/26; A61L 2/24

(52) U.S. Cl. CPC A61L 2/202; A61L 2/26; A61L 2/24; A61L 2202/14; A61L 2202/122; A61L 2202/121; A61L 2202/11

(57) ABSTRACT

A system and a kit for microbial control within an enclosure comprising an electronic component are provided. The enclosure includes an access cover configured to move to enable access to an interior of the enclosure with the electronic component at least partially positioned within the interior of the enclosure. An oxidant generator is configured to generate an oxidizing agent in a gaseous state and distribute the oxidizing agent in the gaseous state within the interior of the enclosure. The oxidant generator may be positioned within the interior of the enclosure, or the oxidant generator may be in fluid communication with the interior of the enclosure. The oxidant generator may be an ozone generator, such as an ultraviolet (UV) light source or an electrical discharge source, and the oxidizing agent may be ozone. Alternatively, the oxidant generator may be a chlorine dioxide generator, and the oxidizing agent may be chlorine dioxide.

### CROSS-REFERENCE TO RELATED APPLICATIONS

This application claims priority to U.S. Provisional Patent Application No. 62/678,645, filed May 31, 2018, which is assigned to the assignee hereof and hereby expressly incorporated by reference herein in its entirety as if fully set forth below and for all applicable purposes.

### BACKGROUND

### Technical Field

Aspects of the present disclosure relate to microbial control within an enclosure including one or more electronic components, especially an enclosure used within a processing facility.

### Description of the Related Art

Electrical panels or enclosures including one or more components, such as electronic components, can harbor undesirable bacteria and other microorganisms (i.e., microbes). This is particularly harmful in food processing plants, medical manufacturing facilities, and cosmetics manufacturing facilities. For example, although an electronic enclosure is typically not a primary food contact surface, the enclosure does have the potential to indirectly transfer microorganisms to food products within a food processing plant. The electronic enclosure may be hardened to tolerate sanitation and exterior washing. However, even with these precautions, the electronic enclosure is still capable of creating an environment capable of generating microbial growth, in which case such microorganisms could be unintentionally transferred from the electronic enclosure to one or more primary food contact surfaces within the food processing plant.

Similarly, while an electronic enclosure is typically not in contact with products (e.g., pharmaceuticals or makeup) of a medical manufacturing facility or a cosmetics manufacturing facility, for example, the enclosure does have the potential to indirectly transfer microorganisms to products within a processing facility (e.g., a factory or laboratory) in these and other such industries.

### SUMMARY

The devices, apparatuses, systems, and methods of this disclosure each have several aspects, no single one of which is solely responsible for its desirable attributes. Without limiting the scope of this disclosure as expressed by the claims which follow, some features will now be discussed briefly. After considering this discussion, and particularly after reading the section entitled “Detailed Description” one will understand how the features of this disclosure provide advantages that include improved food safety.

Aspects of the present disclosure generally relate to microbial control within an enclosure including one or more electronic components, especially an enclosure used within a processing facility, such as a food processing facility, a manufacturing facility for medicines, or a cosmetics manufacturing facility.

Certain aspects of the present disclosure provide a microbial control system for use in a processing facility. The microbial control system generally includes an enclosure with an access cover configured to be selectively opened to enable access to an interior of the enclosure, an electronic component at least partially disposed in the interior of the enclosure, wherein the electronic component is configured to interact with the processing facility, and an oxidant generator configured to generate an oxidizing agent in a gaseous state and distribute the oxidizing agent in the gaseous state within the interior of the enclosure.

Certain aspects of the present disclosure provide a microbial control system for use in a processing facility. The microbial control system generally includes an enclosure with an access cover configured to be selectively opened to enable access to an interior of the enclosure, an electronic component at least partially disposed in the interior of the enclosure, wherein the electronic component is configured to interact with the processing facility, and an oxidant generator configured to generate an oxidizing agent in a gaseous state and distribute the oxidizing agent in the gaseous state within the interior of the enclosure, wherein the oxidant generator is positioned within the interior of the enclosure.

Certain aspects of the present disclosure provide a microbial control system for use in a processing facility. The microbial control system generally includes an enclosure with an access cover configured to be selectively opened to enable access to an interior of the enclosure, an electronic component at least partially disposed in the interior of the enclosure, wherein the electronic component is configured to interact with the processing facility, and an oxidant generator configured to generate an oxidizing agent in a gaseous state and distribute the oxidizing agent in the gaseous state within the interior of the enclosure, wherein the oxidant generator comprises an ozone generator and wherein the oxidizing agent comprises ozone.

Certain aspects of the present disclosure provide a microbial control system for use in a processing facility. The microbial control system generally includes an enclosure with an access cover configured to be selectively opened to enable access to an interior of the enclosure, an electronic component at least partially disposed in the interior of the enclosure, wherein the electronic component is configured to interact with the processing facility, and an oxidant generator configured to generate an oxidizing agent in a gaseous state and distribute the oxidizing agent in the gaseous state within the interior of the enclosure, wherein the oxidant generator comprises an ultraviolet (UV) light source and wherein the oxidizing agent comprises ozone.

Certain aspects of the present disclosure provide a microbial control system for use in a processing facility. The microbial control system generally includes an enclosure with an access cover configured to be selectively opened to enable access to an interior of the enclosure, an electronic component at least partially disposed in the interior of the enclosure, wherein the electronic component is configured to interact with the processing facility, and an oxidant generator configured to generate an oxidizing agent in a gaseous state and distribute the oxidizing agent in the gaseous state within the interior of the enclosure, wherein the oxidant generator comprises an ultraviolet (UV) light source comprising a mercury lamp or a light-emitting diode (LED) and wherein the oxidizing agent comprises ozone.

Certain aspects of the present disclosure provide a microbial control system for use in a processing facility. The microbial control system generally includes an enclosure with an access cover configured to be selectively opened to enable access to an interior of the enclosure, an electronic component at least partially disposed in the interior of the enclosure, wherein the electronic component is configured to interact with the processing facility, and an oxidant generator configured to generate an oxidizing agent in a gaseous state and distribute the oxidizing agent in the gaseous state within the interior of the enclosure, wherein the oxidant generator comprises an ozone generator that comprises an electrical discharge source having a pair of electrodes configured to generate an electric spark in a gap between the pair of electrodes and wherein the oxidizing agent comprises ozone.

Certain aspects of the present disclosure provide a microbial control system for use in a processing facility. The microbial control system generally includes an enclosure with an access cover configured to be selectively opened to enable access to an interior of the enclosure, an electronic component at least partially disposed in the interior of the enclosure, wherein the electronic component is configured to interact with the processing facility, and an oxidant generator configured to generate an oxidizing agent in a gaseous state and distribute the oxidizing agent in the gaseous state within the interior of the enclosure, wherein the oxidant generator comprises a chlorine dioxide generator and wherein the oxidizing agent comprises chlorine dioxide.

Certain aspects of the present disclosure provide a microbial control system for use in a processing facility. The microbial control system generally includes an enclosure with an access cover configured to be selectively opened to enable access to an interior of the enclosure, an electronic component at least partially disposed in the interior of the enclosure, wherein the electronic component is configured to interact with the processing facility, and an oxidant generator configured to generate an oxidizing agent in a gaseous state and distribute the oxidizing agent in the gaseous state within the interior of the enclosure, wherein the oxidant generator comprises a chlorine dioxide generator that comprises a tablet configured to interact with an acid or water to generate the oxidizing agent that comprises chlorine dioxide.

Certain aspects of the present disclosure provide a microbial control system for use in a processing facility. The microbial control system generally includes an enclosure with an access cover configured to be selectively opened to enable access to an interior of the enclosure, an electronic component at least partially disposed in the interior of the enclosure, wherein the electronic component is configured to interact with the processing facility, and an oxidant generator configured to generate an oxidizing agent in a gaseous state and distribute the oxidizing agent in the gaseous state within the interior of the enclosure, wherein the oxidant generator comprises a chlorine dioxide generator that comprises a chlorite configured to interact with an acid to generate the oxidizing agent that comprises chlorine dioxide.

Certain aspects of the present disclosure provide a microbial control system for use in a processing facility. The microbial control system generally includes an enclosure with an access cover configured to be selectively opened to enable access to an interior of the enclosure, an electronic component at least partially disposed in the interior of the enclosure, wherein the electronic component is configured to interact with the processing facility, and an oxidant generator configured to generate an oxidizing agent in a gaseous state and distribute the oxidizing agent in the gaseous state within the interior of the enclosure, wherein the oxidant generator comprises a chlorine dioxide generator that comprises at least one of sodium chlorite or potassium chlorite configured to interact with at least one of hydrochloric acid or sulfuric acid to generate the oxidizing agent that comprises chlorine dioxide.

Certain aspects of the present disclosure provide a microbial control system for use in a processing facility. The microbial control system generally includes an enclosure with an access cover configured to be selectively opened to enable access to an interior of the enclosure, an electronic component at least partially disposed in the interior of the enclosure, wherein the electronic component is configured to interact with the processing facility, an oxidant generator configured to generate an oxidizing agent in a gaseous state and distribute the oxidizing agent in the gaseous state within the interior of the enclosure, and a switch operably coupled to the enclosure such that the switch is in a first position when the access cover is open with respect to the enclosure and the switch is in a second position when the access cover is in a closed position with respect to the enclosure wherein the oxidant generator is operably coupled to the switch such that the switch is configured to at least one of: cause the oxidant generator to operate when the switch is in the second position; or prevent operation of the oxidant generator when the switch is in the first position.

Certain aspects of the present disclosure provide a microbial control system for use in a processing facility. The microbial control system generally includes an enclosure with an access cover configured to be selectively opened to enable access to an interior of the enclosure, an electronic component at least partially disposed in the interior of the enclosure, wherein the electronic component is configured to interact with the processing facility, an oxidant generator configured to generate an oxidizing agent in a gaseous state and distribute the oxidizing agent in the gaseous state within the interior of the enclosure, and a sensor positioned within the enclosure and configured to measure a concentration of the oxidizing agent within the interior of the enclosure, wherein the sensor is operably coupled to the oxidant generator such that a signal generated by the sensor is configured to at least one of: prevent the oxidant generator from operating when the concentration is above a first threshold; or cause the oxidant generator to operate when the concentration is below a second threshold.

Certain aspects of the present disclosure provide a microbial control system for use in a processing facility. The microbial control system generally includes an enclosure with an access cover configured to be selectively opened to enable access to an interior of the enclosure, an electronic component at least partially disposed in the interior of the enclosure, wherein the electronic component is configured to interact with the processing facility, an oxidant generator configured to generate an oxidizing agent in a gaseous state and distribute the oxidizing agent in the gaseous state within the interior of the enclosure, and a timer operably coupled to the oxidant generator such that the oxidant generator is configured to operate based on the timer.

Certain aspects of the present disclosure provide a microbial control system for use in a processing facility. The microbial control system generally includes an enclosure with an access cover configured to be selectively opened to enable access to an interior of the enclosure, an electronic component at least partially disposed in the interior of the enclosure, wherein the electronic component is configured to interact with the processing facility, an oxidant generator configured to generate an oxidizing agent in a gaseous state and distribute the oxidizing agent in the gaseous state within the interior of the enclosure, and a controller operably coupled to the oxidant generator and programmed to control the oxidant generator based on at least one of a first signal from a timer, a second signal from a sensor, or a third signal from a user interface.

Certain aspects of the present disclosure provide a microbial control system for use in a processing facility. The microbial control system generally includes an enclosure with an access cover configured to be selectively opened to enable access to an interior of the enclosure, an electronic component at least partially disposed in the interior of the enclosure, wherein the electronic component is configured to interact with the processing facility, and an oxidant generator configured to generate an oxidizing agent in a gaseous state and distribute the oxidizing agent in the gaseous state within the interior of the enclosure, wherein the oxidant generator is configured to receive electrical power from the electronic component.

Certain aspects of the present disclosure provide a microbial control system for use in a processing facility. The microbial control system generally includes an enclosure with an access cover configured to be selectively opened to enable access to an interior of the enclosure, an electronic component at least partially disposed in the interior of the enclosure, wherein the electronic component is configured to interact with the processing facility, and an oxidant generator configured to generate an oxidizing agent in a gaseous state and distribute the oxidizing agent in the gaseous state within the interior of the enclosure, wherein the oxidant generator is configured to receive electrical power from a power source separate from the electronic component.

Certain aspects of the present disclosure provide a microbial control system for use in a processing facility. The microbial control system generally includes an enclosure with an access cover configured to be selectively opened to enable access to an interior of the enclosure, an electronic component at least partially disposed in the interior of the enclosure, wherein the electronic component is configured to interact with the processing facility, and an oxidant generator configured to generate an oxidizing agent in a gaseous state and distribute the oxidizing agent in the gaseous state within the interior of the enclosure, wherein the oxidant generator is configured to receive electrical power from a portable power source separate from the electronic component.

Certain aspects of the present disclosure provide a microbial control system for use in a processing facility. The microbial control system generally includes an enclosure with an access cover configured to be selectively opened to enable access to an interior of the enclosure, an electronic component at least partially disposed in the interior of the enclosure, wherein the electronic component is configured to interact with the processing facility, an oxidant generator configured to generate an oxidizing agent in a gaseous state and distribute the oxidizing agent in the gaseous state within the interior of the enclosure, and a pressure source in fluid communication with the interior of the enclosure and configured to provide a positive pressure in the interior of the enclosure.

Certain aspects of the present disclosure provide a microbial control system for use in a processing facility. The microbial control system generally includes an enclosure with an access cover configured to be selectively opened to enable access to an interior of the enclosure, an electronic component at least partially disposed in the interior of the enclosure, wherein the electronic component is configured to interact with the processing facility, an oxidant generator configured to generate an oxidizing agent in a gaseous state and distribute the oxidizing agent in the gaseous state within the interior of the enclosure, and a pressure source in fluid communication with the interior of the enclosure and configured to provide a positive pressure in the interior of the enclosure, wherein the pressure source comprises a pump that is configured to provide the positive pressure in the interior of the enclosure.

Certain aspects of the present disclosure provide a microbial control system for use in a processing facility. The microbial control system generally includes an enclosure with an access cover configured to be selectively opened to enable access to an interior of the enclosure, an electronic component at least partially disposed in the interior of the enclosure, wherein the electronic component is configured to interact with the processing facility, and an oxidant generator configured to generate an oxidizing agent in a gaseous state and distribute the oxidizing agent in the gaseous state within the interior of the enclosure, wherein the oxidant generator is in fluid communication with the interior of the enclosure.

Certain aspects of the present disclosure provide a microbial control system for use in a processing facility. The microbial control system generally includes an enclosure with an access cover configured to be selectively opened to enable access to an interior of the enclosure, an electronic component at least partially disposed in the interior of the enclosure, wherein the electronic component is configured to interact with the processing facility, an oxidant generator configured to generate an oxidizing agent in a gaseous state and distribute the oxidizing agent in the gaseous state within the interior of the enclosure, and an oxidant generator housing, wherein the oxidant generator is positioned within an interior of the oxidant generator housing and wherein the oxidant generator and the interior of the oxidant generator housing are in fluid communication with the interior of the enclosure.

Certain aspects of the present disclosure provide a microbial control system for use in a processing facility. The microbial control system generally includes an enclosure with an access cover configured to be selectively opened to enable access to an interior of the enclosure, an electronic component at least partially disposed in the interior of the enclosure, wherein the electronic component is configured to interact with the processing facility, an oxidant generator configured to generate an oxidizing agent in a gaseous state and distribute the oxidizing agent in the gaseous state within the interior of the enclosure, an oxidant generator housing, wherein the oxidant generator is positioned within an interior of the oxidant generator housing and wherein the oxidant generator and the interior of the oxidant generator housing are in fluid communication with the interior of the enclosure, and a pressure source in fluid communication with the interior of the oxidant generator housing to provide a positive pressure in the interior of the oxidant generator housing.

Certain aspects of the present disclosure provide a kit for microbial control within an enclosure. The kit includes a switch configured to operably couple to an access cover of the enclosure such that the switch is configured to be in a first position when the access cover is in an open position with respect to the enclosure and the switch is in a second position when the access cover is in a closed position with respect to the enclosure, and an oxidant generator configured to be positioned within an interior of the enclosure, generate an oxidizing agent in a gaseous state within the interior of the enclosure, and be operably coupled to the switch, wherein the switch is configured to at least one of: prevent operation of the oxidant generator when the switch is in the first position; or cause the oxidant generator to operate when the switch is in the second position.

Certain aspects of the present disclosure provide a kit for microbial control within an enclosure. The kit includes a switch configured to operably couple to an access cover of the enclosure such that the switch is configured to be in a first position when the access cover is in an open position with respect to the enclosure and the switch is in a second position when the access cover is in a closed position with respect to the enclosure, an oxidant generator configured to be positioned within an interior of the enclosure, generate an oxidizing agent in a gaseous state within the interior of the enclosure, and be operably coupled to the switch, wherein the switch is configured to at least one of: prevent operation of the oxidant generator when the switch is in the first position; or cause the oxidant generator to operate when the switch is in the second position, and a controller operably coupled to the oxidant generator and programmed to control the oxidant generator based on at least one of the switch, a first signal from a timer, a second signal from a sensor, or a third signal from a user interface.

Certain aspects of the present disclosure provide a kit for microbial control within an enclosure. The kit includes a switch configured to operably couple to an access cover of the enclosure such that the switch is configured to be in a first position when the access cover is in an open position with respect to the enclosure and the switch is in a second position when the access cover is in a closed position with respect to the enclosure, and an oxidant generator configured to be positioned within an interior of the enclosure, generate an oxidizing agent in a gaseous state within the interior of the enclosure, and be operably coupled to the switch, wherein the switch is configured to at least one of: prevent operation of the oxidant generator when the switch is in the first position; or cause the oxidant generator to operate when the switch is in the second position, wherein the oxidant generator is configured to receive electrical power from the electronic component.

Certain aspects of the present disclosure provide a kit for microbial control within an enclosure. The kit includes a switch configured to operably couple to an access cover of the enclosure such that the switch is configured to be in a first position when the access cover is in an open position with respect to the enclosure and the switch is in a second position when the access cover is in a closed position with respect to the enclosure, and an oxidant generator configured to be positioned within an interior of the enclosure, generate an oxidizing agent in a gaseous state within the interior of the enclosure, and be operably coupled to the switch, wherein the switch is configured to at least one of: prevent operation of the oxidant generator when the switch is in the first position; or cause the oxidant generator to operate when the switch is in the second position, wherein the oxidant generator is configured to receive electrical power from a power source separate from the electronic component.

Certain aspects of the present disclosure provide a kit for microbial control within an enclosure. The kit includes a switch configured to operably couple to an access cover of the enclosure such that the switch is configured to be in a first position when the access cover is in an open position with respect to the enclosure and the switch is in a second position when the access cover is in a closed position with respect to the enclosure, an oxidant generator configured to be positioned within an interior of the enclosure, generate an oxidizing agent in a gaseous state within the interior of the enclosure, and be operably coupled to the switch, wherein the switch is configured to at least one of: prevent operation of the oxidant generator when the switch is in the first position; or cause the oxidant generator to operate when the switch is in the second position, and a pressure source configured to be operably coupled to the enclosure such that the pressure source is configured to be in fluid communication with the interior of the enclosure to provide a positive pressure in the interior of the enclosure.

Certain aspects of the present disclosure provide a kit for microbial control within an enclosure. The kit includes a switch configured to operably couple to an access cover of the enclosure such that the switch is configured to be in a first position when the access cover is in an open position with respect to the enclosure and the switch is in a second position when the access cover is in a closed position with respect to the enclosure, and an oxidant generator configured to be positioned within an interior of the enclosure, generate an oxidizing agent in a gaseous state within the interior of the enclosure, and be operably coupled to the switch, wherein the switch is configured to at least one of: prevent operation of the oxidant generator when the switch is in the first position; or cause the oxidant generator to operate when the switch is in the second position, wherein the oxidant generator comprises an ozone generator and wherein the oxidizing agent comprises ozone.

Certain aspects of the present disclosure provide a kit for microbial control within an enclosure. The kit includes a switch configured to operably couple to an access cover of the enclosure such that the switch is configured to be in a first position when the access cover is in an open position with respect to the enclosure and the switch is in a second position when the access cover is in a closed position with respect to the enclosure, and an oxidant generator configured to be positioned within an interior of the enclosure, generate an oxidizing agent in a gaseous state within the interior of the enclosure, and be operably coupled to the switch, wherein the switch is configured to at least one of: prevent operation of the oxidant generator when the switch is in the first position; or cause the oxidant generator to operate when the switch is in the second position, wherein the oxidant generator comprises an ozone generator that comprises an ultraviolet (UV) light source and wherein the oxidizing agent comprises ozone.

Certain aspects of the present disclosure provide a kit for microbial control within an enclosure. The kit includes a switch configured to operably couple to an access cover of the enclosure such that the switch is configured to be in a first position when the access cover is in an open position with respect to the enclosure and the switch is in a second position when the access cover is in a closed position with respect to the enclosure, and an oxidant generator configured to be positioned within an interior of the enclosure, generate an oxidizing agent in a gaseous state within the interior of the enclosure, and be operably coupled to the switch, wherein the switch is configured to at least one of: prevent operation of the oxidant generator when the switch is in the first position; or cause the oxidant generator to operate when the switch is in the second position, wherein the oxidant generator comprises an ozone generator that comprises an ultraviolet (UV) light source comprising a mercury lamp or a light-emitting diode (LED) and wherein the oxidizing agent comprises ozone.

Certain aspects of the present disclosure provide a kit for microbial control within an enclosure. The kit includes a switch configured to operably couple to an access cover of the enclosure such that the switch is configured to be in a first position when the access cover is in an open position with respect to the enclosure and the switch is in a second position when the access cover is in a closed position with respect to the enclosure, and an oxidant generator configured to be positioned within an interior of the enclosure, generate an oxidizing agent in a gaseous state within the interior of the enclosure, and be operably coupled to the switch, wherein the switch is configured to at least one of: prevent operation of the oxidant generator when the switch is in the first position; or cause the oxidant generator to operate when the switch is in the second position, wherein the oxidant generator comprises an ozone generator that comprises an electrical discharge source having a pair of electrodes configured to generate an electric spark in a gap between the pair of electrodes and wherein the oxidizing agent comprises ozone.

Certain aspects of the present disclosure provide a kit for microbial control within an enclosure. The kit includes a switch configured to operably couple to an access cover of the enclosure such that the switch is configured to be in a first position when the access cover is in an open position with respect to the enclosure and the switch is in a second position when the access cover is in a closed position with respect to the enclosure, and an oxidant generator configured to be positioned within an interior of the enclosure, generate an oxidizing agent in a gaseous state within the interior of the enclosure, and be operably coupled to the switch, wherein the switch is configured to at least one of: prevent operation of the oxidant generator when the switch is in the first position; or cause the oxidant generator to operate when the switch is in the second position, wherein the oxidant generator comprises a chlorine dioxide generator and wherein the oxidizing agent comprises chlorine dioxide.

Certain aspects of the present disclosure provide a kit for microbial control within an enclosure. The kit includes a switch configured to operably couple to an access cover of the enclosure such that the switch is configured to be in a first position when the access cover is in an open position with respect to the enclosure and the switch is in a second position when the access cover is in a closed position with respect to the enclosure, and an oxidant generator configured to be positioned within an interior of the enclosure, generate an oxidizing agent in a gaseous state within the interior of the enclosure, and be operably coupled to the switch, wherein the switch is configured to at least one of: prevent operation of the oxidant generator when the switch is in the first position; or cause the oxidant generator to operate when the switch is in the second position, wherein the oxidant generator comprises a chlorine dioxide generator that comprises a tablet configured to interact with an acid or water to generate the oxidizing agent that comprises chlorine dioxide.

Certain aspects of the present disclosure provide a kit for microbial control within an enclosure. The kit includes a switch configured to operably couple to an access cover of the enclosure such that the switch is configured to be in a first position when the access cover is in an open position with respect to the enclosure and the switch is in a second position when the access cover is in a closed position with respect to the enclosure, and an oxidant generator configured to be positioned within an interior of the enclosure, generate an oxidizing agent in a gaseous state within the interior of the enclosure, and be operably coupled to the switch, wherein the switch is configured to at least one of: prevent operation of the oxidant generator when the switch is in the first position; or cause the oxidant generator to operate when the switch is in the second position, wherein the oxidant generator comprises a chlorine dioxide generator that comprises a chlorite configured to interact with an acid to generate the oxidizing agent that comprises chlorine dioxide.

Certain aspects of the present disclosure provide a kit for microbial control within an enclosure. The kit includes a switch configured to operably couple to an access cover of the enclosure such that the switch is configured to be in a first position when the access cover is in an open position with respect to the enclosure and the switch is in a second position when the access cover is in a closed position with respect to the enclosure, and an oxidant generator configured to be positioned within an interior of the enclosure, generate an oxidizing agent in a gaseous state within the interior of the enclosure, and be operably coupled to the switch, wherein the switch is configured to at least one of: prevent operation of the oxidant generator when the switch is in the first position; or cause the oxidant generator to operate when the switch is in the second position, wherein the oxidant generator comprises a chlorine dioxide generator that comprises at least one of sodium chlorite and potassium chlorite configured to interact with at least one of hydrochloric acid or sulfuric acid to generate the oxidizing agent that comprises chlorine dioxide.

Certain aspects of the present disclosure provide a method for controlling microbes. The method generally includes receiving a signal indicative of an access cover of an enclosure being closed to block access to an interior of the enclosure and controlling an oxidant generator, based on reception of the signal, to introduce an oxidizing agent into the interior of the enclosure.

Certain aspects of the present disclosure provide a method for controlling microbes. The method generally includes receiving a signal indicative of an access cover of an enclosure being closed to block access to an interior of the enclosure, controlling an oxidant generator, based on reception of the signal, to introduce an oxidizing agent into the interior of the enclosure, receiving a signal indicative of the access cover being open to permit access to the interior of the enclosure, and controlling the oxidant generator to stop operation in response to the signal indicative of the access cover being open.

Certain aspects of the present disclosure provide a method for controlling microbes. The method generally includes receiving a signal indicative of an access cover of an enclosure being closed to block access to an interior of the enclosure, controlling an oxidant generator, based on reception of the signal, to introduce an oxidizing agent into the interior of the enclosure, causing the oxidant generator to generate the oxidizing agent in a gaseous state, and causing the oxidizing agent in the gaseous state to be distributed within the interior of the enclosure.

Certain aspects of the present disclosure provide a method for controlling microbes. The method generally includes receiving a signal indicative of an access cover of an enclosure being closed to block access to an interior of the enclosure and controlling an oxidant generator, based on reception of the signal, to introduce an oxidizing agent into the interior of the enclosure, wherein the oxidant generator comprises an ozone generator and wherein the oxidizing agent comprises ozone.

Certain aspects of the present disclosure provide a method for controlling microbes. The method generally includes receiving a signal indicative of an access cover of an enclosure being closed to block access to an interior of the enclosure and controlling an oxidant generator, based on reception of the signal, to introduce an oxidizing agent into the interior of the enclosure, wherein the oxidant generator comprises an ozone generator that comprises an ultraviolet (UV) light source and wherein the oxidizing agent comprises ozone.

Certain aspects of the present disclosure provide a method for controlling microbes. The method generally includes receiving a signal indicative of an access cover of an enclosure being closed to block access to an interior of the enclosure and controlling an oxidant generator, based on reception of the signal, to introduce an oxidizing agent into the interior of the enclosure, wherein the oxidant generator comprises an ozone generator that comprises an ultraviolet (UV) light source that comprises a mercury lamp or a light-emitting diode (LED) and wherein the oxidizing agent comprises ozone.

Certain aspects of the present disclosure provide a method for controlling microbes. The method generally includes receiving a signal indicative of an access cover of an enclosure being closed to block access to an interior of the enclosure and controlling an oxidant generator, based on reception of the signal, to introduce an oxidizing agent into the interior of the enclosure, wherein the oxidant generator comprises an ozone generator that comprises an electrical discharge source having a pair of electrodes configured to generate an electric spark in a gap between the pair of electrodes and wherein the oxidizing agent comprises ozone.

Certain aspects of the present disclosure provide a method for controlling microbes. The method generally includes receiving a signal indicative of an access cover of an enclosure being closed to block access to an interior of the enclosure and controlling an oxidant generator, based on reception of the signal, to introduce an oxidizing agent into the interior of the enclosure, wherein the oxidant generator comprises a chlorine dioxide generator and wherein the oxidizing agent comprises chlorine dioxide.

Certain aspects of the present disclosure provide a method for controlling microbes. The method generally includes receiving a signal indicative of an access cover of an enclosure being closed to block access to an interior of the enclosure and controlling an oxidant generator, based on reception of the signal, to introduce an oxidizing agent into the interior of the enclosure, wherein the oxidant generator comprises a chlorine dioxide generator that comprises a tablet configured to interact with an acid or water to generate the oxidizing agent that comprises chlorine dioxide.

Certain aspects of the present disclosure provide a method for controlling microbes. The method generally includes receiving a signal indicative of an access cover of an enclosure being closed to block access to an interior of the enclosure and controlling an oxidant generator, based on reception of the signal, to introduce an oxidizing agent into the interior of the enclosure, wherein the oxidant generator comprises a chlorine dioxide generator that comprises a chlorite configured to interact with an acid to generate the oxidizing agent that comprises chlorine dioxide.

Certain aspects of the present disclosure provide a method for controlling microbes. The method generally includes receiving a signal indicative of an access cover of an enclosure being closed to block access to an interior of the enclosure and controlling an oxidant generator, based on reception of the signal, to introduce an oxidizing agent into the interior of the enclosure, wherein the oxidant generator comprises a chlorine dioxide generator that comprises at least one of sodium chlorite or potassium chlorite configured to interact with at least one of hydrochloric acid or sulfuric acid to generate the oxidizing agent that comprises chlorine dioxide.

Certain aspects of the present disclosure provide a method for controlling microbes. The method generally includes receiving a signal indicative of an access cover of an enclosure being closed to block access to an interior of the enclosure, controlling an oxidant generator, based on reception of the signal, to introduce an oxidizing agent into the interior of the enclosure, and determining a concentration of the oxidizing agent in the interior of the enclosure, wherein controlling the oxidant generator further comprises controlling the oxidant generator based on the determined concentration of the oxidizing agent in the interior of the enclosure.

Certain aspects of the present disclosure provide a method for controlling microbes. The method generally includes receiving a signal indicative of an access cover of an enclosure being closed to block access to an interior of the enclosure and controlling an oxidant generator, based on reception of the signal and a timer, to introduce an oxidizing agent into the interior of the enclosure.

Certain aspects of the present disclosure provide a method for controlling microbes. The method generally includes receiving a signal indicative of an access cover of an enclosure being closed to block access to an interior of the enclosure and controlling an oxidant generator, based on reception of the signal and another signal from a user input device, to introduce an oxidizing agent into the interior of the enclosure.

Certain aspects of the present disclosure provide a method for controlling microbes. The method generally includes receiving a signal indicative of an access cover of an enclosure being closed to block access to an interior of the enclosure, controlling an oxidant generator, based on reception of the signal, to introduce an oxidizing agent into the interior of the enclosure, and controlling a positive pressure device to provide a positive pressure in the interior of the enclosure.

Certain aspects of the present disclosure provide a method for controlling microbes. The method generally includes receiving a signal indicative of an access cover of an enclosure being closed to block access to an interior of the enclosure, controlling an oxidant generator, based on reception of the signal, to introduce an oxidizing agent into the interior of the enclosure, and controlling a positive pressure device to provide a positive pressure in the interior of the enclosure, wherein the oxidant generator is positioned within an interior of an oxidant generator housing in fluid communication with the interior of the enclosure and wherein controlling the positive pressure device comprises controlling the positive pressure device to provide the positive pressure within the interior of the oxidant generator housing.

Aspects of the present disclosure generally include methods, apparatus, and systems, as substantially described herein with reference to and as illustrated by the accompanying drawings. Numerous other aspects are provided.

To the accomplishment of the foregoing and related ends, the one or more aspects comprise the features hereinafter fully described and particularly pointed out in the claims. The following description and the annexed drawings set forth in detail certain illustrative features of the one or more aspects. These features are indicative, however, of but a few of the various ways in which the principles of various aspects may be employed, and this description is intended to include all such aspects and their equivalents.

### BRIEF DESCRIPTION OF THE DRAWINGS

So that the manner in which the above-recited features of the present disclosure can be understood in detail, a more particular description, briefly summarized above, may be had by reference to aspects, some of which are illustrated in the appended drawings. It is to be noted, however, that the appended drawings illustrate only certain typical aspects of this disclosure and are therefore not to be considered limiting of its scope, for the description may admit to other equally effective aspects.

FIG. 1 is a schematic perspective view of a microbial control system in accordance with certain aspects of the present disclosure.

FIG. 2 is a schematic cutaway view of the microbial control system shown in FIG. 1.

FIG. 3 is a schematic view of a microbial control system with a controller and sources of various signals, in accordance with certain aspects of the present disclosure.

FIG. 4 is a schematic view of a microbial control system with one or more power sources, in accordance with certain aspects of the present disclosure.

FIG. 5 is a schematic view of a microbial control system positioned outside of an enclosure, in accordance with certain aspects of the present disclosure.

FIGS. 6A-6D are schematic views of exemplary oxidant generators used to generate ozone, in accordance with certain aspects of the present disclosure.

FIGS. 7A-7C are schematic views of exemplary oxidant generators used to generate chlorine dioxide, in accordance with certain aspects of the present disclosure.

FIG. 8 is a flow diagram illustrating example operations for controlling microbes, in accordance with certain aspects of the present disclosure.

To facilitate understanding, identical reference numerals have been used, where possible, to designate identical elements that are common to the figures. It is contemplated that elements described in one aspect may be beneficially utilized on other aspects without specific recitation.

### DETAILED DESCRIPTION

Aspects of the present disclosure provide apparatus, systems, and methods for microbial control within an enclosure including one or more electronic components within a processing facility. One example microbial control system generally includes an enclosure comprising an access cover configured to be selectively opened to enable access to an interior of the enclosure; an electronic component at least partially disposed in the interior of the enclosure, wherein the electronic component is operable to interact with the processing facility; and an oxidant generator configured to generate an oxidizing agent in a gaseous state and distribute the oxidizing agent in the gaseous state within the interior of the enclosure.

The following description provides examples, and is not limiting of the scope, applicability, or examples set forth in the claims. Changes may be made in the function and arrangement of elements discussed without departing from the scope of the disclosure. Various examples may omit, substitute, or add various procedures or components as appropriate. For instance, the methods described may be performed in an order different from that described, and various steps may be added, omitted, or combined. Also, features described with respect to some examples may be combined in some other examples. For example, an apparatus may be implemented or a method may be practiced using any number of the aspects set forth herein. In addition, the scope of the disclosure is intended to cover such an apparatus or method which is practiced using other structure, functionality, or structure and functionality in addition to or other than the various aspects of the disclosure set forth herein. It should be understood that any aspect of the disclosure described herein may be embodied by one or more elements of a claim. The word “exemplary” is used herein to mean “serving as an example, instance, or illustration.” Any aspect described herein as “exemplary” is not necessarily to be construed as preferred or advantageous over other aspects.

As shown and described herein, various features of the disclosure will be presented. Various aspects may have the same or similar features and thus the same or similar features may be labeled with the same reference numeral. Although similar reference numbers may be used in a generic sense, various aspects will be described and various features may include changes, alterations, modifications, etc., as will be appreciated by those of skill in the art, whether explicitly described or otherwise.

Electronic component enclosures are increasing in quantity and quality (e.g., sophistication) as electronic components increase in use in modern society. For example, as the internet of things continues to grow, along with the level and sophistication of automation, so will the number of electronic component enclosures. Controlling the growth and exposure to microorganisms with respect to these electronic component enclosures becomes increasingly important, particularly in industries for food processing, medical applications, and for manufacturing products such as drugs, dietary supplements, medical materials, or consumables.

Further, factors facilitating microbial growth are enhanced with the potential variable temperatures and environment for the electronic component enclosures. For example, cold temperatures used within a food processing environment may cause a box to “breathe” as temperatures vary with air and gas being drawn into and expelled from the electronic component enclosure. This movement of gas into and out of the electronic component enclosure increases the ability for microorganisms to transfer into and out of the electronic component enclosure. Adding moisture and/or using the electronic component enclosures within a wet environment also adds another risk factor for facilitating microbial growth. The food processing environment often incorporates both cold and wet environments, and electronic component enclosures are often opened periodically during use, introducing even further risk.

Thus, aspects of the present disclosure generally relate to microbial control within a system including an enclosure. The enclosure includes an interior with one or more electronic components positioned within the interior of the enclosure. The electronic component may include a distribution board (panelboard, breaker panel, or electric panel), a semiconductor component, an electronic circuit, an integrated circuit, a power converter (e.g., voltage regulator), and/or one or more other types of electronic components. The enclosure includes an access feature (e.g., a door or access panel) that is movable (or removable) to enable access to the interior of the enclosure, such as for accessing and interacting with the electronic component. Further, an oxidant generator is in use with the enclosure, such as to generate an oxidizing agent and distribute the oxidizing agent within the interior of the enclosure. The oxidant generator may be used to generate the oxidizing agent in a gaseous state for distribution within the interior of the enclosure. The oxidant generator may be positioned within the interior of the enclosure. Alternatively, the oxidant generator may be positioned external to the interior of the enclosure, but may be in fluid communication with the interior of the enclosure, such as to have the oxidizing agent in the gaseous form routed to the interior of the enclosure from a separate housing or location. The oxidizing agent is able to interact with microorganisms to oxidize, control, and kill the microorganisms. Thus, microbes, such as listeria and/or mold, may be controlled and prevented from growth by distributing the oxidizing agent within the interior of the enclosure.

### Examples of Microbial Control for an Enclosure

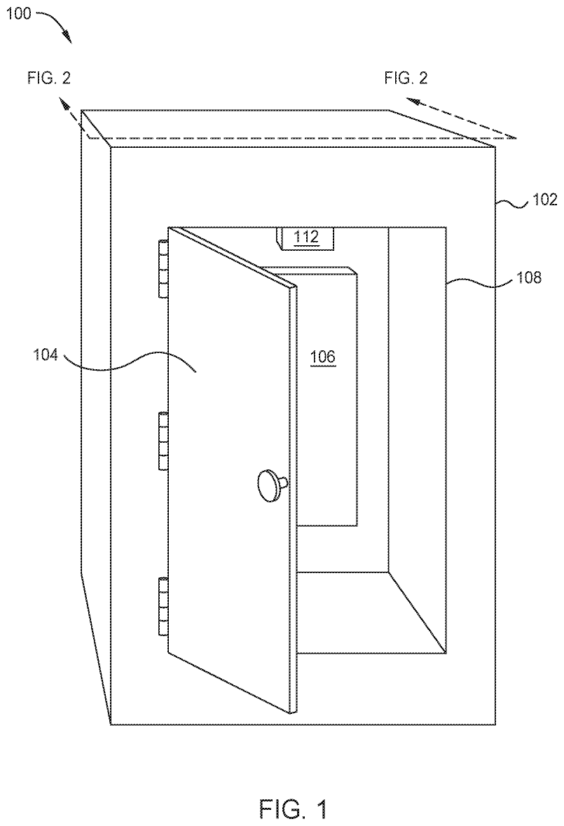

Referring now to FIG. 1, a schematic view of a microbial control system 100 in accordance with one or more aspects of the present disclosure is shown. The system 100 may be used within any system or environment (e.g., a process facility) where it is desired to control microorganisms within an enclosure 102. Accordingly, the system 100 may be used within a food processing system, a medical application system, or another system, such as for processing products that include drugs, dietary supplements, medical materials, or consumables. The system 100 includes the enclosure 102 with an access cover 104 (e.g., a door or sliding access panel) movable (or removable) to enable access to an interior of the enclosure 102. An electronic component 106 is at least partially positioned within the interior of the enclosure 102 such that the electronic component 106 is at least partially housed within and protected by the enclosure 102. Although only one electronic component 106 is shown in FIG. 1, the reader is to understand that there may be more than one electronic component disposed within the interior of the enclosure 102. An example of an electronic component 106 may include a controller of an Automated SmartWash Analytical Platform (ASAP)™, available from SmartWash Solutions, LLC of Salinas, Calif., and described within U.S. Patent Application Publication No. 2018/0093901 to Brennan et al., filed on Oct. 3, 2017 and entitled “System for Controlling Water Used for Industrial Food Processing,” which is incorporated by reference herein in its entirety. The access cover 104 enables access to the electronic component 106 within the interior of the enclosure 102, such as when interacting with the electronic component 106, for example, for maintaining or replacing the electronic component 106.

The access cover 104 is movable (or removable) between an open position and a closed position with respect to the enclosure 102. The open position for the access cover 104 is shown in FIG. 1. In the open position, the access cover 104 enables access to the interior of the enclosure 102 through an opening 108, such as for interacting with the electronic component 106. In the closed position, the access cover 104 may be secured to the enclosure 102 to enclose and seal the interior of the enclosure 102 and prevent access to the interior of the enclosure 102.

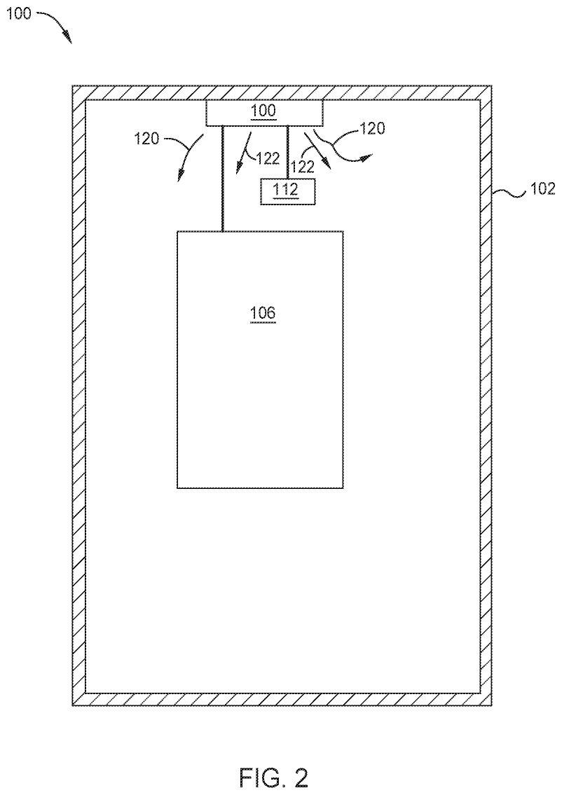

The system 100 may further include a switch 112 that is operably coupled to the access cover 104 such that the switch is in a first position when the access cover 104 is in the open position with respect to the opening 108 of the enclosure 102 and the switch is in a second position when the access cover 104 is in a closed position with respect to the opening 108. As discussed in more detail below with reference to FIG. 2, when the switch 112 is in the first position, an oxidant generator in the enclosure 102 is prevented from operating, and when the switch 112 is in the second position, the oxidant generator in the enclosure 102 may operate. An example of the switch 112 may include a switch relay, such as a double-pole switch relay. The switch 112 may be positioned adjacent the access cover 104 and/or the opening 108 to measure a position of the access cover 104 with respect to the opening 108 and/or the enclosure 102. In another aspect, as the access cover 104 may be rotatable with respect to the enclosure 102 to move between the open position and the closed position (e.g., in the case of a door), the switch 112 may be able to measure the amount of rotation between the access cover 104 and the enclosure 102. Further, in another aspect, a latch or lock may be used with the access cover 104 to secure the access cover 104 in the closed position with respect to the opening 108 of the enclosure 102. In such an aspect, the switch 112 may be operably coupled to the latch or lock such that the switch is in the first position when the access cover 104 is in the closed position, but not secured in the closed position with the latch or lock. That is, the switch 112 may be in the first position that prevents an oxidant generator in the enclosure 102 from operating, when the access cover 104 is closed, but not secured with the latch. When both the access cover 104 is the closed position and the latch or lock is in the secured position, the switch 112 may be in the second position, allowing or causing the oxidant generator to operate. Furthermore, in another aspect, the switch 112 may be able to detect a presence of external light, such as natural light, being received into the interior of the enclosure 102. In such an aspect, the switch 112 may be in the first position if external light is received within the enclosure 102.

FIG. 2 is a schematic cutaway view of the microbial control system 100, in accordance with aspects of the present disclosure. An oxidant generator 110 is included with the microbial control system 100. In FIG. 2, the oxidant generator 110 is shown positioned within the interior of the enclosure 102 to distribute the oxidizing agent within the interior of the enclosure 102. The oxidant generator 110 is used to generate one or more oxidizing agents 120 in a gaseous state and then distribute the one or more oxidizing agents 120 within the interior of the enclosure 102. The one or more oxidizing agents 120 from the oxidant generator 110 may be used to oxidize existing microorganisms included within the enclosure 102, and/or may be used to prevent growth of microorganisms within the enclosure 102, thereby preventing the enclosure 102 with the electronic component 106 from being a potential microbial source that may contaminate a larger system or facility that uses the enclosure 102 with the electronic component 106. For certain aspects, the oxidant generator 110 may also generate electromagnetic radiation (e.g., ultraviolet (UV) light) 122 that may kill or inactivate microorganisms in the enclosure 102.

Generating the oxidizing agent 120 in a gaseous state, as performed by the oxidant generator 110, may enable the antimicrobial properties of the oxidizing agent to be distributed within the enclosure 102 and may be suitable for the safety of a system or facility incorporating the electronic component 106, such as a food processing system or other system where microbial contamination should be avoided (e.g., a system for processing drugs, medical materials, or cosmetics). In one aspect, the oxidizing agent 120 includes ozone such that the oxidant generator 110 is an ozone generator to generate ozone. In another aspect, the oxidizing agent 120 includes chlorine dioxide such that the oxidant generator 110 is a chlorine dioxide generator to generate chlorine dioxide.

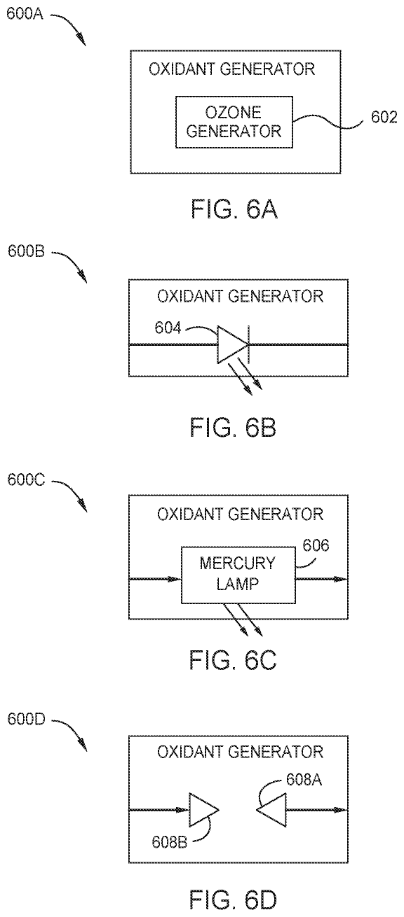

In one aspect, ozone may be generated from infusing energy with oxygen in the air. Further, after ozone dissipates, the ozone may leave substantially no residue behind. FIGS. 6A-6D show various examples of oxidant generators 600A-600D that may be used to generate ozone and may be considered examples of the oxidant generator 110 illustrated in FIG. 2. In FIG. 6A, as discussed, the oxidant generator 600A may include an ozone generator 602 used to generate ozone. In one or more aspects, ozone may be generated at a low but lethal level for microbial control from ultraviolet (UV) light or an electrical discharge. Thus, an ozone generator may include a UV light source and/or an electrical discharge source. FIG. 6B shows an example of a UV light source for an oxidant generator 600B that includes at least one light-emitting diode (LED) 604 for generating UV light. FIG. 6C shows an example of a UV light source for an oxidant generator 600C that includes a mercury lamp 606 for generating UV light. Other examples of UV light sources may also be used for an ozone generator, such as other types of lamps or bulbs, without departing from the scope of the present disclosure.

As discussed, the UV light source generates electromagnetic radiation that may interact with oxygen in the air to create ozone. Further, the UV light source itself, in addition to the ozone created by the UV light source, may have antimicrobial properties to kill microorganisms. For example, though the antimicrobial properties of the UV light are limited to areas in a line of sight from the UV light source and thus those microorganisms that are shaded from the UV light source may remain unaffected, ozone generated by the UV light source may be able to kill and destroy microorganisms that are shaded from the UV light source.

An example is shown in FIG. 6D of an oxidant generator 600D including an electrical discharge source. In this aspect, the electrical discharge source may include one or more electrodes, such as a pair of electrodes 608. An electric spark may be generated in the gap between the pair of the electrodes 608, in which the energy of the electrical discharge may interact with oxygen in the air to create ozone. This type of arrangement with a pair of electrodes 608 separated by a gap may be referred to as a “spark gap.”

In one or more aspects of the present disclosure and based upon several factors, such as the amount of ozone being generated and/or the size of the interior of the enclosure 102, the UV light source may use between about five watts to about twenty-five watts of electrical power, and more specifically about six watts of electrical power. Further, the UV light source may produce UV light having a wavelength between about 10 nm and 400 nm, and more specifically about 240 nm. A UV light source of this power level may be able to sanitize and control microorganisms in a ten cubic foot (10 ft³) enclosure in less than about one hour (e.g., 52 minutes). If the treatment is continuous from the UV light source, or any oxidant generator 110 in general, microorganisms, and listeria specifically, may not be able to form colonies in the enclosure 102. If the enclosure 102 is rarely opened, a timer, discussed in more detail below, may be used to reduce power consumption and/or extend the lifetime of the oxidant generator 110. For example, a UV light source may have an operating life of about 10,000 hours, so a timer may extend the useful life of the UV light source by causing the UV light source to be on often enough to prevent microorganisms from forming colonies in the enclosure while preventing the UV light source from continuously operating.

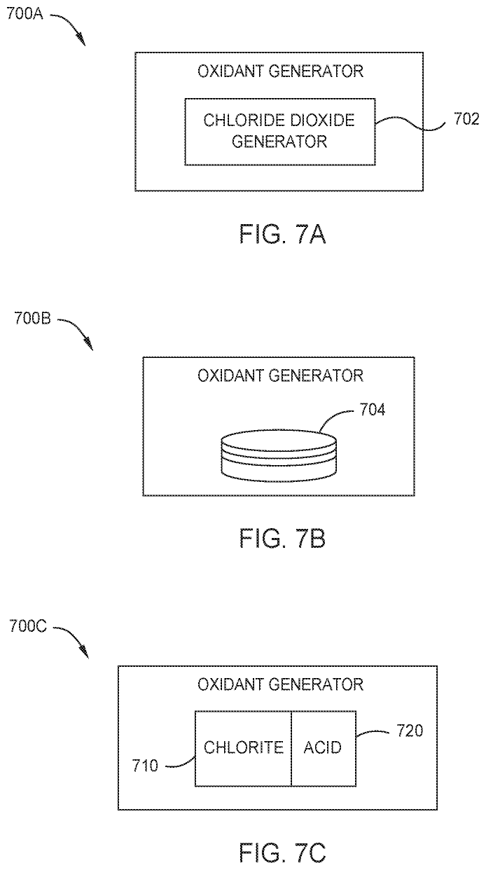

The oxidant generator 110 may additionally or alternatively include a chlorine dioxide generator to generate chlorine dioxide. FIGS. 7A-7C show various examples of oxidant generators 700A-700C that may be used to generate chlorine dioxide. In FIG. 7A, the oxidant generator 700A may include a chlorine dioxide generator 702 used to generate chlorine dioxide. In one or more aspects, the chlorine dioxide generator 702 may be configured to cause multiple chemicals to react with each other and generate chlorine dioxide.

FIG. 7B shows an example of a chemical component used for, or as a part of, an oxidant generator 700B in the form of a tablet 704. The tablet 704 may include or be formed from a chlorite, such as sodium chlorite or potassium chlorite. The tablet 704 may interact with another chemical, such as acid or water, to generate chlorine dioxide. The tablet 704 may be released from an inert storage (e.g., from a sealed bag) to begin reacting with another chemical manually, such as by an operator removing the tablet 704 from a sealed package and positioning the tablet 704 upon a holder or tray within an oxidant generator. The tablet 704 may alternatively be released automatically, such as by having a motor open a cover of a sealed chamber or compartment containing the tablet 704 to release the tablet 704 and cause the tablet 704 to be exposed to or interact with acid or water. The acid or water may also be released manually or automatically, similar to the tablet 704. Further, the tablet 704 may be able to interact with moisture in the air to generate chlorine dioxide, as opposed to having to introduce the water separately.

FIG. 7C shows a schematic of an exemplary oxidant generator 700C including chlorite 710 used to interact with acid 720 to generate chlorine dioxide. The chlorite, which may be in the form of the tablet 704 (shown in FIG. 7B), may interact with acid, such as hydrochloric acid or sulfuric acid, to generate chlorine dioxide.

Returning to FIG. 2, the oxidant generator 110 may be operably coupled to the switch 112 such that the operation of the oxidant generator 110 may be controlled based upon the state of the switch 112. For example, in one aspect, the switch 112 may be in a first position when the access cover 104 (see FIG. 1) is in the closed position and/or when the access cover 104 is secured in the closed position, and the switch 112 may be in a second position when the access cover 104 is in the open position or when the access cover 104 is not secured (e.g., not latched) in the closed position. In the example, the switch 112 may prevent the oxidant generator 110 from operating when the switch is in the second position (e.g., when the access cover 104 is in the open position or the access cover 104 is not secured in the closed position). In such an aspect, the switch 112 may be operably coupled to the access cover 104, to the opening 108, or to a securing mechanism (e.g., a latch or lock) coupled to the access cover 104 or the enclosure 102. This control of the operation of the oxidant generator 110 may increase the effectiveness of the microbial control for the oxidant generator 110 within the enclosure 102, and may provide a safety barrier for those that interact with the enclosure 102.

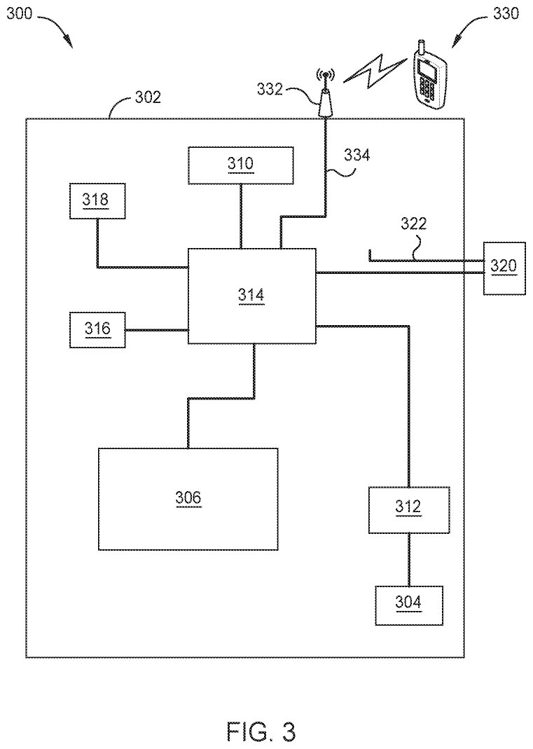

Referring now to FIG. 3, a schematic view of an exemplary microbial control system 300 in accordance with one or more aspects of the present disclosure is shown. The system 300 includes an enclosure 302 with an access cover 304 movable (or altogether removable) to enable access to an interior of the enclosure 302 and at least one electronic component 306 positioned within the interior of the enclosure 302. An example of an electronic component 306 may include a controller of an Automated SmartWash Analytical Platform (ASAP)™, available from SmartWash Solutions, LLC of Salinas, Calif., and described within U.S. Patent Application Publication No. 2018/0093901 to Brennan et al., filed on Oct. 3, 2017 and entitled “System for Controlling Water Used for Industrial Food Processing.” An oxidant generator 310 is also positioned within the interior of the enclosure 302 to distribute the oxidizing agent within the interior of the enclosure 302. A switch 312 is also included with the system 300 by being operably coupled to the oxidant generator 310.

Further, the system 300 includes a controller 314 operably coupled to the oxidant generator 310 with the controller 314 including or being operably coupled to one or more other components. As shown, the controller 314, which may be a programmable logic controller (PLC), for example, is operably coupled to the electronic component 306 and the switch 312, and may also be operably coupled to (or include) a timer 316, a sensor 318, an antenna 332, and/or a user interface 330. The user interface 330 may wirelessly communicate with the controller 314 via an antenna 332 that is coupled to the controller via a wire 334 and a transceiver (not shown), or alternatively, the user interface 330 may be connected to the controller via a wire (not shown). For example, the timer 316, which may be a timer relay, may generate a timer signal (e.g., a first signal) to control the operation of the oxidant generator 310. Additionally or alternatively, the sensor 318 may generate a sensor signal (e.g., a second signal) to control the operation of the oxidant generator 310, and/or the user interface 330 may generate a user input signal (e.g., a third signal) to control the operation of the oxidant generator 310 via the antenna 332 and wire 334. The controller 314 may be operably coupled between the electronic component 306, the oxidant generator 310, the switch 312, the timer 316, the sensor 318, the antenna 332, and/or the user interface 330 and may be programmed to control the oxidant generator 310 based on a switch signal from the switch 312, the timer signal from the timer 316, the sensor signal from the sensor 318, and/or the user input signal from the user interface 330 or antenna 332. As the controller 314 is operably coupled to the electronic component 306, the oxidant generator 310, the switch 312, the timer 316, the sensor 318, the antenna 332, and/or the user interface 330, the controller 314 may be wired and/or wirelessly connected with each of these components to facilitate communication and control therebetween.

As shown, the sensor 318 may be positioned within the enclosure 302 and may be used to measure the oxidizing agent within the interior of the enclosure 302. The sensor 318 may be used to measure the presence of the oxidizing agent within the enclosure 302 and/or the amount or concentration of the oxidizing agent within the enclosure 302. The sensor 318 (e.g., in conjunction with the controller 314) may be used to control the operation of the oxidant generator 310 and/or may be able to determine if the oxidant generator 310 is working properly. For example, the oxidizing agent may be generated and distributed by the oxidant generator 310 within the interior of the enclosure 302 at a predetermined rate or at a predetermined concentration. The sensor 318 may be used to verify or control the oxidant generator 310 based upon a comparison of the measured rate or concentration of the oxidizing agent within the interior of the enclosure 302 and the predetermined rate, the predetermined concentration, or a threshold (e.g., a minimum or a maximum) concentration.

Further, the oxidizing agent may be a dangerous agent, such that for those (e.g., facility personnel) working in proximity to the oxidizing agent, the amount or level of oxidizing agent is moderated or even regulated in the work place by the Occupational Safety and Health Administration (OSHA). The oxidizing agent may soften plastic and/or insulation by breaking down polymers, and thus may also be destructive for the enclosure and/or the electronic component within the enclosure. Thus, it is desirable that the oxidant generator provide a quantity of oxidizing agent sufficient to sanitize and control the microorganisms within the enclosure 302, but not so much that the oxidizing agent damages the enclosure 302 and/or related equipment, possibly resulting in a premature failure. Thus, the sensor 318 may be used to facilitate monitoring of the oxidizing agent produced by the oxidant generator 310.

Referring still to FIG. 3, a pressure source 320, such as a pump, may be operably coupled with the interior of the enclosure 302 to provide a positive pressure in the interior of the enclosure 302 for certain aspects. The pressure source may generate a positive pressure (e.g., pumping air, nitrogen, or another gas into the enclosure 302) by providing pressure into the interior of the enclosure 302 to generate a higher pressure within the interior of the enclosure 302 than a pressure exterior to the enclosure 302. The pressure source 320 may be positioned within, or partially within, the enclosure 302 to provide the positive pressure within the enclosure 302. Alternatively, the pressure source 320 may be positioned exterior to the enclosure 302 with the pressure source in fluid communication with the interior of the enclosure 302. The pressure source 320 may be in fluid communication with the interior of the enclosure 302 by having the gas routed through a flow line 322 (e.g., a conduit or pipe), as shown, to provide the positive pressure from the pressure source to the interior of the enclosure 302. Further, the pressure source 320 may be operably coupled to the controller 314, as shown, such that the controller 314 is programmed to control the operation of the pressure source. For example, the controller 314 may be used to control the operation of the pressure source 320 based upon the operation of the oxidant generator 310 such that the pressure source 320 and the oxidant generator 310 operate concurrently or overlap in their operating times.

The pressure source 320 may be used to create a positive pressure environment within the interior of the enclosure 302. A positive pressure environment may facilitate microbial control within the enclosure 302, such as by preventing air or another gas from entering the interior of the enclosure 302, due to the pressure difference between the interior and the exterior of the enclosure 302 causing air and other fluids to flow out of the enclosure 302 and not into the enclosure 302. In one aspect, the pressure source 320 may create a positive pressure of about four inches of water pressure. Further, depending on the size of the enclosure 302, the pressure source may be able to pump air or gas at about one to two cubic feet per hour into the interior of the enclosure 302. Furthermore, the pressure source 320 may provide gas pressure through the oxidant generator 310 to facilitate distribution of the oxidizing agent within the interior of the enclosure 302. For example, gas pressure from the pressure source 320 may be provided between the pair of electrodes 608 (shown in FIG. 6D) to distribute the ozone from the pair of electrodes 608 within the interior of the enclosure 302.

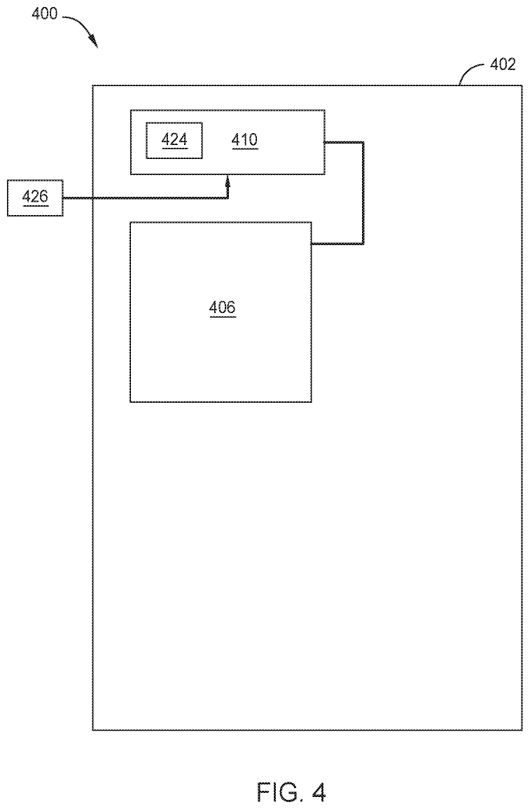

Referring now to FIG. 4, a schematic view of an exemplary microbial control system 400 in accordance with one or more aspects of the present disclosure is shown. The system 400 includes an enclosure 402 with an electronic component 406 positioned (at least partially) within the interior of the enclosure 402. An example of an electronic component 406 may include a controller of an Automated SmartWash Analytical Platform (ASAP)™, available from SmartWash Solutions, LLC of Salinas, Calif., and described within U.S. Patent Application Publication No. 2018/0093901 to Brennan et al., filed on Oct. 3, 2017 and entitled “System for Controlling Water Used for Industrial Food Processing.” An oxidant generator 410 is included with the system 400 to distribute an oxidizing agent within the interior of the enclosure 402. As shown, if the oxidant generator 410 requires electrical power for operation, the oxidant generator 410 may receive electrical power from one or more sources, such as at about 120 volts of alternating-current power (VAC) at 60 Hz, 110 VAC at 50 Hz, or about 24 volts of direct-current power (VDC). For example, with reference to FIG. 4, the oxidant generator 410 may receive electrical power from the electronic component 406, and more specifically from a voltage regulator or other power supply circuit in the electronic component 406.

The oxidant generator 410 may additionally or alternatively receive electrical power from a power source separate from the electronic component 406, such as from an internal power source 424 and/or from an external power source 426. The internal power source 424 may be positioned within the enclosure 402 and/or may be included within the oxidant generator 410. The internal power source 424 may be portable, such as a battery. Further, the internal power source 424 may be rechargeable. The external power source 426 may be external to the enclosure 402. The external power source 426 may be portable or non-portable, and in one or more aspects, the external power source 426 may be used to charge the internal power source 424.

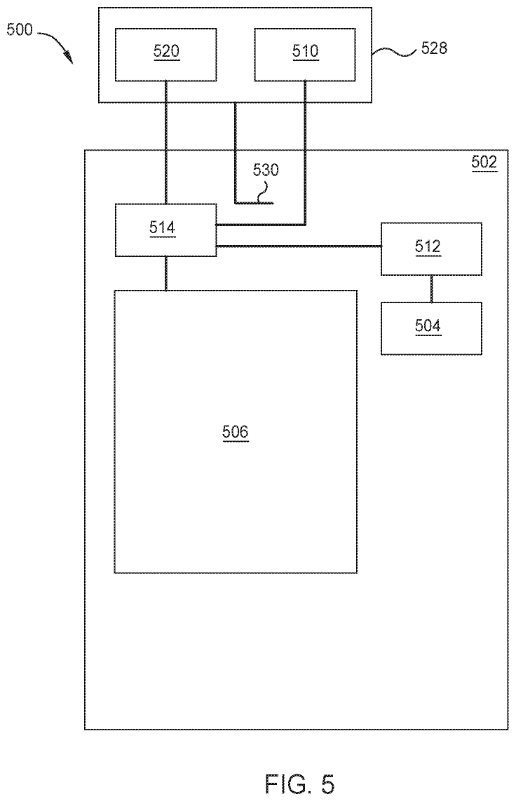

Referring now to FIG. 5, a schematic view of an exemplary microbial control system 500 in accordance with one or more aspects of the present disclosure is shown. As with the above aspects, the system 500 includes an enclosure 502 with an electronic component 506 positioned (at least partially) within the interior of the enclosure 502. An example of an electronic component 506 may include a controller of an Automated SmartWash Analytical Platform (ASAP)™, available from SmartWash Solutions, LLC of Salinas, Calif., and described within U.S. Patent Application Publication No. 2018/0093901 to Brennan et al., filed on Oct. 3, 2017 and entitled “System for Controlling Water Used for Industrial Food Processing.” The system 500 also includes an oxidant generator 510 to generate an oxidizing agent and distribute the oxidizing agent within the interior of the enclosure 502. However, in this aspect, rather than having the oxidant generator 510 positioned within the enclosure 502, the oxidant generator 510 is positioned exterior to the enclosure 502 and is in fluid communication with the interior of the enclosure 502.

For example, the system 500 may further include an oxidant generator housing 528 with the oxidant generator 510 positioned within an interior of the oxidant generator housing 528. The oxidant generator housing 528 may be in fluid communication with the interior of the enclosure 502, such as through a flow line 530 (e.g., a tube or pipe), such that the oxidizing agent generated by the oxidant generator 510 is distributed to the interior of the enclosure 502 through the flow line 530. Further, a pressure source 520, such as a pump, may be used to provide a positive pressure to the interior of the oxidant generator housing 528, so as to facilitate fluid communication and pumping of the oxidizing agent from the oxidant generator housing 528 to the enclosure 502. As shown in FIG. 5, the pressure source 520 may be positioned within the interior of the oxidant generator housing 528 and provide the positive pressure to the interior of the enclosure 502 through the flow line 530.

The exemplary microbial control system 500 may optionally include a controller 514 and a switch 512. As described above with reference to FIG. 3, controller may control operation of the oxidant generator 510 based on a signal from the switch 512. The switch 512 may generate a signal based on a position of an access cover 504, which may be closed to prevent access to the interior of the enclosure 502. The controller 514 may also control operation of the pressure source 520, based on the signal from the switch 512. The controller 514 may further receive other signals from a sensor within the enclosure 502 and/or a user interface and control the oxidant generator 510 and pressure source 520 based on those other signals.

In one or more other aspects, an oxidant generator in accordance with the present disclosure may include one or more other chemical or physical sources for microbial control. For example, other chemicals having antimicrobial properties, in addition or as an alternative to oxidizing agents such as ozone and/or chlorine dioxide discussed above, may be used. Further, a heat source may be included within an oxidant generator for microbial control, such as by generating thermal energy to cause a temperature within the enclosure to be above a predetermined temperature (e.g., a threshold temperature), such that microorganisms cannot live within the enclosure.

One or more aspects of the present disclosure may be used to retrofit an existing enclosure including an electronic component, such as to introduce microbial control for the enclosure. Aspects of the present disclosure may include providing a kit or group of parts that may be used for microbial control for an existing enclosure. The kit may include a switch configured to operably couple to an access cover of the enclosure such that the switch is in a first position when the access cover is in an open position with respect to an opening of the enclosure and the switch is in a second position when the access cover is in a closed position with respect to the opening, as described above. The kit may further include an oxidant generator, such as a UV light source, that is positionable within an interior of the enclosure. The oxidant generator of the kit may generate an oxidizing agent in a gaseous state for use within the interior of the enclosure and be operably coupled to the switch such that the switch causes the oxidant generator to operate when the switch is in a certain position (e.g., indicating the access cover is in the closed position). Further, the kit may include a controller and/or a pressure source. The controller may be operably coupled to the oxidant generator and programmed to control the oxidant generator based upon the switch, a first signal from a timer, a second signal from a sensor, and/or a third signal from a user interface. The pressure source may be configured to be placed in fluid communication with the interior of the enclosure to provide a positive pressure in the interior of the enclosure.

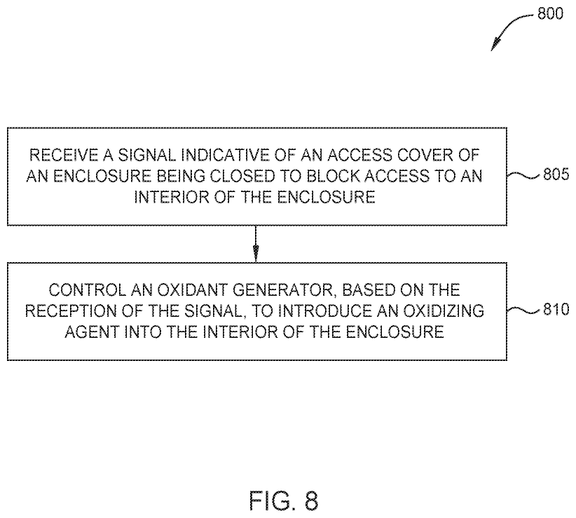

FIG. 8 is a flow diagram illustrating example operations 800 for controlling microbes, in accordance with certain aspects of the present disclosure. The operations 800 may be performed, for example, by a controller (e.g., controller 314, shown in FIG. 3) of a microbial control system, such as the microbial control system 300 shown in FIG. 3.

The operations 800 may begin, at block 805, with receiving a signal indicative of an access cover of an enclosure being closed to block access to an interior of the enclosure. For example, controller 314 (see FIG. 3) may receive a signal from switch 312 indicative of an access cover (e.g., a door) of the enclosure 302 being closed to block access to an interior of the enclosure 302.

At block 810, the operations 800 continue with controlling an oxidant generator, based on reception of the signal, to introduce an oxidizing agent into the interior of the enclosure. Continuing the example from above, the controller 314 (see FIG. 3) may control the oxidant generator 310, based on reception of a signal from the switch 312. The oxidant generator may be disposed in the enclosure or external to the enclosure.

Aspects in accordance with the present disclosure may be able to improve microbial control in enclosures, particularly for enclosures used within a microbial sensitive environment, such as within the food processing industry, the medical application industry, or the cosmetics industry. Aspects in accordance with the present disclosure may include electronic components positioned wholly or partially within the enclosure, but may also include or alternatively have other components commonly positioned within enclosures, such as mechanical components (e.g., valves or a manifold). Further, a microbial control system may be included with a motor control panel or enclosure, such as a variable drive motor control panel or enclosure, a logic controller panel or enclosure, a power distribution panel or enclosure, a process equipment control panel or enclosure, and/or a wash line or instrument control panel or enclosure (e.g., disclosed in U.S. Patent Application Publication No. 2018/0093901, entitled “SYSTEM FOR CONTROLLING WATER USED FOR INDUSTRIAL FOOD PROCESSING,” filed on Oct. 3, 2017, and incorporated by reference herein in its entirety).

For example, an enclosure or a system capable of using an enclosure within the food processing industry may incorporate one or more aspects of the present disclosure. An enclosure may include one or more elements for controlling, testing, or detecting one or more substances used within a food processing system, such as controlling water chemistry (e.g., monitoring and controlling pH level and/or chlorine level for water used within a food processing system). These elements may include a sensor, a pump, a valve, a controller and/or a processor, and a human machine interface (HMI), such as a video display screen, to display information to a user. One or more of these elements may be positioned within the enclosure, and the enclosure may be portable, so as to be moved within a food processing plant, or may be non-portable and fixed in place (e.g., fixed to a larger structure). Certain aspects of the present disclosure may be incorporated within an enclosure used within a food processing system, such as by being retrofitted to be included within or operable with the enclosure. An oxidant generator, such as a UV light source, may be positioned within the interior of the enclosure to generate and distribute an oxidizing agent within the enclosure. A switch and a pump may be included and operable with the oxidant generator. Further, the oxidant generator may be electrically coupled to one or more pre-existing elements within the enclosure to receive electrical power. Thus, the present disclosure contemplates other elements and uses in addition or as alternatives to those provided and discussed above.

While the present disclosure has been described in detail in connection with a limited number of aspects, it should be readily understood that the present disclosure is not limited to such described aspects. Rather, the present disclosure can be modified to incorporate any number of variations, alterations, substitutions, combinations, sub-combinations, or equivalent arrangements not heretofore described, but which are commensurate with the scope of the present disclosure. Additionally, while various aspects of the present disclosure have been described, it is to be understood that aspects of the present disclosure may include only some of the described features.

The term “about” is intended to include the degree of error associated with measurement of the particular quantity based upon the equipment available at the time of filing the application. For example, “about” can include a range of ±8%, 5%, or 2% of a given value.

The terminology used herein is for the purpose of describing particular aspects only and is not intended to be limiting of the present disclosure. As used herein, the singular forms “a,” “an,” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, element components, and/or groups thereof.

While the present disclosure has been described with reference to exemplary aspects, it will be understood by those skilled in the art that various changes may be made and equivalents may be substituted for elements thereof without departing from the scope of the present disclosure. In addition, many modifications may be made to adapt a particular situation or material to the teachings of the present disclosure without departing from the essential scope thereof.

Therefore, it is intended that the present disclosure not be limited to the particular aspect or aspects included as the best mode contemplated for carrying out the present disclosure, but that the present disclosure will include all aspects falling within the scope of the claims.

What is claimed is:

1. A microbial control system for use in a processing facility, comprising:
an enclosure comprising an access cover configured to be selectively opened to enable access to an interior of the enclosure;
an electronic component at least partially disposed in the interior of the enclosure, wherein the electronic component is configured to interact with the processing facility; and
an oxidant generator configured to generate an oxidizing agent in a gaseous state and distribute the oxidizing agent in the gaseous state within the interior of the enclosure.

2. The microbial control system of claim 1, wherein the oxidant generator is positioned within the interior of the enclosure.

3. The microbial control system of claim 1, wherein the oxidant generator comprises an ozone generator and wherein the oxidizing agent comprises ozone.

4. The microbial control system of claim 3, wherein the ozone generator comprises an ultraviolet (UV) light source.

5. The microbial control system of claim 1, wherein the oxidant generator comprises a chlorine dioxide generator and wherein the oxidizing agent comprises chlorine dioxide.

6. The microbial control system of claim 1, further comprising:
a switch operably coupled to the enclosure such that the switch is in a first position when the access cover is open with respect to the enclosure and the switch is in a second position when the access cover is in a closed position with respect to the enclosure wherein the oxidant generator is operably coupled to the switch such that the switch is configured to at least one of:
cause the oxidant generator to operate when the switch is in the second position; or
prevent operation of the oxidant generator when the switch is in the first position.

7. The microbial control system of claim 1, further comprising a sensor positioned within the enclosure and configured to measure a concentration of the oxidizing agent within the interior of the enclosure, wherein the sensor is operably coupled to the oxidant generator such that a signal generated by the sensor is configured to at least one of:
prevent the oxidant generator from operating when the concentration is above a first threshold; or
cause the oxidant generator to operate when the concentration is below a second threshold.

8. The microbial control system of claim 1, further comprising a timer operably coupled to the oxidant generator such that the oxidant generator is configured to operate based on the timer.

9. The microbial control system of claim 1, further comprising a controller operably coupled to the oxidant generator and programmed to control the oxidant generator based on at least one of a first signal from a timer, a second signal from a sensor, or a third signal from a user interface.

10. The microbial control system of claim 1, wherein the oxidant generator is configured to receive electrical power from the electronic component.

11. The microbial control system of claim 1, wherein the oxidant generator is configured to receive electrical power from a power source separate from the electronic component.

12. The microbial control system of claim 1, further comprising a pressure source in fluid communication with the interior of the enclosure and configured to provide a positive pressure in the interior of the enclosure.

13. The microbial control system of claim 1, wherein the oxidant generator is in fluid communication with the interior of the enclosure.

14. A kit for microbial control within an enclosure, comprising:
a switch configured to operably couple to an access cover of the enclosure or another component of the enclosure such that the switch is configured to be in a first position when the access cover is in an open position with respect to the enclosure and the switch is configured to be in a second position when the access cover is in a closed position with respect to the enclosure; and
an oxidant generator configured to:
be positioned within an interior of the enclosure;
generate an oxidizing agent in a gaseous state within the interior of the enclosure; and
be operably coupled to the switch, wherein the switch is further configured to at least one of:
prevent operation of the oxidant generator when the switch is in the first position; or
cause the oxidant generator to operate when the switch is in the second position.

15. The kit of claim 14, further comprising a controller operably coupled to the oxidant generator and programmed to control the oxidant generator based on at least one of the switch, a first signal from a timer, a second signal from a sensor, or a third signal from a user interface.

16. The kit of claim 14, wherein the oxidant generator is configured to receive electrical power from the electronic component.

17. The kit of claim 14, wherein the oxidant generator is configured to receive electrical power from a power source separate from the electronic component.

18. The kit of claim 14, further comprising a pressure source configured to be operably coupled to the enclosure such that the pressure source is configured to be in fluid communication with the interior of the enclosure to provide a positive pressure in the interior of the enclosure.

19. The kit of claim 14, wherein the oxidant generator comprises an ozone generator and wherein the oxidizing agent comprises ozone.

20. The kit of claim 19, wherein the ozone generator comprises an ultraviolet (UV) light source.

21. The kit of claim 14, wherein the oxidant generator comprises a chlorine dioxide generator and wherein the oxidizing agent comprises chlorine dioxide.

22. A method for controlling microbes, comprising:
receiving a signal indicative of an access cover of an enclosure being closed to block access to an interior of the enclosure; and
controlling an oxidant generator, based on reception of the signal, to introduce an oxidizing agent into the interior of the enclosure.

23. The method of claim 22, further comprising:
receiving a signal indicative of the access cover being open to permit access to the interior of the enclosure; and
controlling the oxidant generator to stop operation in response to the signal indicative of the access cover being open.

24. The method of claim 22, wherein controlling the oxidant generator comprises:
causing the oxidant generator to generate the oxidizing agent in a gaseous state; and
causing the oxidizing agent in the gaseous state to be distributed within the interior of the enclosure.

25. The method of claim 22, wherein the oxidant generator comprises an ozone generator and wherein the oxidizing agent comprises ozone.

26. The method of claim 22, wherein the oxidant generator comprises a chlorine dioxide generator and wherein the oxidizing agent comprises chlorine dioxide.

27. The method of claim 22, further comprising determining a concentration of the oxidizing agent in the interior of the enclosure, wherein controlling the oxidant generator further comprises controlling the oxidant generator based on the determined concentration of the oxidizing agent in the interior of the enclosure.

28. The method of claim 22, wherein controlling the oxidant generator further comprises controlling the oxidant generator based on a timer.

29. The method of claim 22, wherein controlling the oxidant generator further comprises controlling the oxidant generator based on a signal from a user input device.

30. The method of claim 22, further comprising controlling a positive pressure device to provide a positive pressure in the interior of the enclosure.
